(19) United States
(12) Patent Application Publication
(10) Pub. No.: US 2022/0168251 A1
(43) Pub. Date: Jun. 2, 2022

(54) PROCESS FOR PRODUCTION OF A NANO-MICROEMULSION SYSTEM OF FERULIC

(71) Applicant: NHAT HAI NEW TECHNOLOGY JOINT STOCK COMPANY, Hanoi (VN)

(72) Inventor: Minh Hai LUU, Hanoi (VN)

(73) Assignee: NHAT HAI NEW TECHNOLOGY JOINT STOCK COMPANY, Hanoi (VN)

(21) Appl. No.: 17/369,235

(22) Filed: Jul. 7, 2021

(30) Foreign Application Priority Data
Dec. 2, 2020 (VN) 1-2020-06983

(51) Int. Cl. A61K 31/192; A61K 9/107; A61K 47/10; A61K 47/26

(52) U.S. Cl. CPC A61K 31/192; A61K 47/26; A61K 47/10; A61K 9/1075

(57) ABSTRACT

Provided is a process for production of a nano-microemulsion system of ferulic acid includes the steps of: a) preparing a dispersal phase of ferulic; b) preparing a carrier mixture of plurolic/tween 80; c) mixing the ferulic dispersal phase and the plurolic/tween 80 carrier mixture; d) removing insoluble substances in the mixture by high-speed centrifugation; e) homogenizing the mixture obtained after the homogenization; and f) filtering the homogeneous mixture obtained after the homogenization.

### CROSS REFERENCE TO RELATED APPLICATIONS

This application claims priority to Vietnam patent application No. 1-2020-06983 filed on Dec. 2, 2020, the entire contents of which are hereby incorporated herein by reference.

### TECHNICAL FIELD

The present invention relates to a process for production of a nano-microemulsion system of ferulic.

### BACKGROUND OF THE PRESENT INVENTION

Ferulic acid which is an abundant phenolic phytochemical found in plant cell walls is an antioxidant and anti-cancer agent. Additionally, the compound is also capable of absorbing UV-rays and lowering blood cholesterol level. Nowadays, ferulic acid is widely used in cosmetic industry, as a food additive and has been increasingly studied and applied in medical field. Ferulic acid is used to prevent or treat certain health conditions such as Alzheimer's disease, atherosclerosis, cancer, high cholesterol, high blood pressure, diabetes, etc.

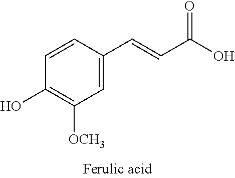 [SMILES: COc1cc(/C=C/C(=O)O)ccc1O]

In 2013, study in Journal of Cardiovascular Pharmacology reported that ferulic acid may help lower blood pressure. The study tested on hypertensive rats treated with ferulic acid for 8 weeks showing a significant decrease in systolic blood pressure, with an improvement in blood flow to kidney and a decrease in left ventricular stiffness in heart (where blood is pumped into body).

Ferulic acid is capable of controlling diabetes better through reducing inflammation in pancreas (where insulin is produced) and liver (where sugar is stored in blood). According to a study in European Journal of Pharmacology (2012), a combination of ferulic acid and resveratrol (another plant-based antioxidant) increased activity of a compound, so-called nuclear factor-kB (NF-kB), in rats. The NF-kB plays a central role in cell inflammation. By reducing the inflammation in the liver and pancreas, scientists concluded that the combination of the compounds showed remarkable anti-diabetic properties.

Several studies on animals suggested ferulic acid may improve cognitive function in people with Alzheimer's disease by reducing oxidative stress that promotes regeneration of brain cells. According to a 2019 study in Journal of Biological Chemistry, genetically engineered rats to develop brain amyloidosis were treated with a combination of ferulic acid and a plant-derived compound, called epigallocatechin gallate (EGCG). After 15 months of the treatment, initial results showed that the combination was able to reverse cognitive impairment (memory and learning) in the rats.

A number of published studies showed that ferulic acid had many valuable biological activities. Such a compound is however susceptible to oxidation and reduction by external environmental agents such as light, temperature and other oxidizing agents. Furthermore, ferulic acid is slightly soluble in water. Due to the above-mentioned disadvantages, ferulic has been studied and converted into highly bioavailable forms, wherein a study of Ujjawal Bairagi et. al. should be included. The study increased the bioavailability of ferulic by encapsulating ferulic in nanoparticles of poly(lactic-co-glycolic acid) (PLGA). The results showed that the nanoparticles had a size of 24 nm, with a ferulic level of about 88%. The advantage of the method is that it is simple and does not require complicated equipments, and the level of ferulic is highly included. The disadvantage of the method is that the nanoparticle sizes are quite large.

Atefe Rezaei et. al. used encapsulation to encapsulate ferulic in cyclodextrin nanoparticles. The resulting nanoparticles had particle a size of more than 360 nm. The method increases the solubility of ferulic acid, but the nanoparticle size is usually quite large.

Preedia et. al. prepared ferulic acid in nano form ZnONPs-FAC in research of toxicity of the product to cancer cells Huh-7 and HepG2. The granulation of over 100 nm was carried out. The method has the advantage of simplicity, increasing the bioavailability of ferulic. However, the microemulsion usually have a short-term stability.

The published studies with different ferulic application orientations and different methods of production have their own advantages and disadvantages, but the included solutions have produced ferulic nano forms with high bioavailability. To increase the bioavailability of ferulic and the stability of ferulic products, the present invention provides a process for production of a nano-microemulsion system of ferulic with long-term stability and high bioavailability. The present pre-microemulsion system is a mixture of oil phases, surfactants, and co-surfactants that can form a nano-emulsion in the gastrointestinal and gastrointestinal environment after oral administration. The emulsion formed after the oral administration includes micro-emulsion parts of less than 100 nm size that are highly soluble in water, thereby increasing absorption of the drug in the gastrointestinal tract. The present pre-microemulsion system has the advantage of stable physical and chemical properties, long-term storage. With the above-mentioned advantages, the present process for production of the pre-microemulsion system is currently being used in food, pharmaceutical and cosmetic technologies.

### SUMMARY OF THE PRESENT INVENTION

An object of the present invention is to provide preparations of ferulic with high solubility, improved membrane permeability, stable activity, and high durability of the pre-emulsion system.

To achieve the above-mentioned object, the present invention provides a process for production of a nano-microemulsion system of ferulic acid, including the steps of:

a) preparing a dispersal phase by dissolving ferulic acid in acrysol K140 in a ratio (g/g) of ferulic/acrysol K140 is 1:4 on a stirrer at 500 rpm for 45 minutes at 40° C. to obtain the ferulic dispersal phase;

b) preparing a carrier mixture of plurolic/tween 80 by mixing plurolic F68 and tween 80 in a ratio (g/g) of plurolic F68/tween 80 is 1:2 on a stirrer at 400 rpm for 2 hours at 60° C.;

c) forming a homogeneous mixture by mixing the ferulic dispersal phase and the carrier mixture of plurolic/tween 80 so that the substances form the homogeneous mixture having a ratio (g/g) of the dispersion/the carrier mixture is 2:5 on a stirrer at 1000 rpm, for 5 hours at 60° C.;

d) removing insoluble substances in the mixture by high-speed centrifugation of 10000 rpm, for 20 minutes at room temperature;

e) leaving the mixture obtained in step d) overnight, subsequently homogenizing in a homogenizing stirrer at 3000 rpm for 1 hour and repeating 5 times; and

f) filtering the homogeneous mixture obtained after the homogenization by a nanofiltration system to obtain the nano-microemulsion system of ferulic.

### BRIEF DESCRIPTION OF THE DRAWINGS

FIG. 1 shows high-resolution transmission electron microscopy (HR-TEM) image of the present ferulic nanoparticles.

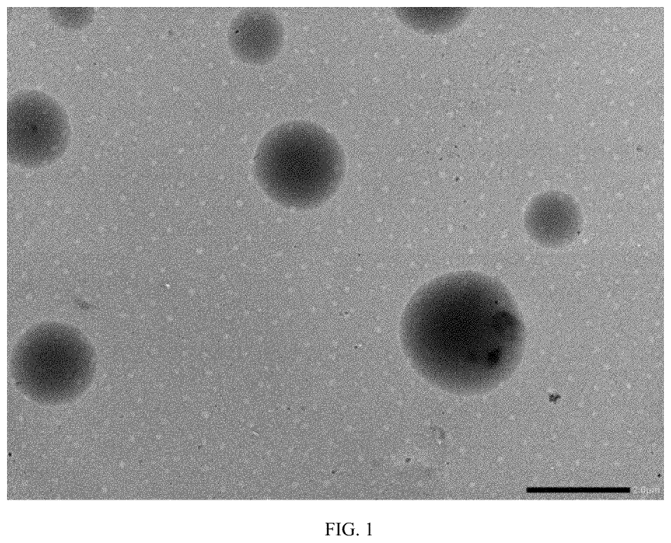

### DETAILED DESCRIPTION OF THE PRESENT INVENTION

Set forth hereafter is a detailed description of preferred embodiments of the present invention, it is to be understood that the disclosure is only illustrative and exemplary of the present disclosure and the detailed description of one or more embodiments is not intended, nor is to be construed, to limit the scope of the present invention.

The present invention provides a process for production of a nano-microemulsion system of ferulic acid, including the steps of:

a) preparing a dispersal phase by dissolving ferulic acid in acrysol K140 in a ratio (g/g) of ferulic/acrysol K140 is 1:4 on a stirrer at 500 rpm for 45 minutes at 40° C. to obtain the ferulic dispersal phase;

Acrysol K140 (also known as PEG-40 hydrogenated castor oil) is a compound of PEG40 and castor oil. Acrysol K140 is capable of increasing solubility of poorly water-soluble pharmaceutical agents. Therefore, acrysol K14 is widely used in pharmaceutical industry. By many experiments, the present ratio (g/g) of ferulic to acrysol K140 is 1:4 was chosen.

b) preparing a carrier mixture of plurolic/tween 80 by mixing plurolic F68 and tween 80 in a ratio (g/g) of plurolic F68/tween 80 is 1:2 on a stirrer at 400 rpm for 2 hours at 60° C.;

Plurolic 68 (also known as Polyoxyethylene-polyoxypropylene block copolymer) is a PEO—PPO—PEO co-polymer of poly(ethylene oxide) (PEO) or poly(propylene oxide) (PPO). It is a non-toxic synthetic polymer that does not exhibit antigenic properties, and has a suitable oil-water partition coefficient. Pluronic has been approved by the Pharmacopoeia of many countries as an adjuvant for many different types of drugs such as oral and topical ones. Tween, a substance made up of reaction of sorbitan fatty acid ester (a non-ionic surfactant) with ethylene oxide, is used in many countries, including the US and EU, where it acts as an emulsifier, dissolving agent in many kinds of food products, including breads, cake mixes, salad dressings, shortening oils and chocolate. Tween 80 is a hydrophilic non-ionic surfactant. It is used as a surfactant in soaps and in cosmetics and as a lubricant in eye drops. In food or medicine, the compound can act as an emulsifier. Tween 80 is an adjuvant used to stabilize medicinal formulations of water used for injection or vaccination. The dissolving agent acts as a surfactant and increases solubility of one agent in another. The pluronic/tween ratios are tested for proper HLB (Hydrophilic-lipophilic balance).

c) forming a homogeneous mixture by mixing the ferulic dispersal phase and the carrier mixture of plurolic/tween 80 so that the substances form the homogeneous mixture having a ratio (g/g) of the dispersion/the carrier mixture is 2:5 on a stirrer at 1000 rpm, for 5 hours at 60° C.;

The dissolving agent/carrier ratio has been tested to ensure the homogeneous mixture without phase separation. By many experiments, the ratio (g/g) of ferulic/plurolic-tween 80 is 2:5, which is selected to form the homogeneous and stable mixture.

d) removing insoluble substances in the mixture by high-speed centrifugation of 10000 rpm, for 20 minutes at room temperature;

e) leaving the mixture obtained in step d) overnight, subsequently homogenizing in a homogenizing stirrer at 3000 rpm for 1 hour and repeating 5 times;

By assimilation with the repeated cycle, the pharmaceutical substance is evenly dispersed in the carrier to form the homogeneous mixture with high stability.

f) filtering the homogeneous mixture obtained after the homogenization by a nanofiltration system to obtain the nano-microemulsion system of ferulic.

### EXAMPLES

### Example 1: Production of 50 g of Pre-Nanoemulsion System of Ferulic

A dispersal phase was prepared by dissolving 3 g of ferulic acid in 12 g of acrysol K140 on a stirrer at 500 rpm for 45 minutes at 40° C. to obtain the dispersal phase of ferulic.

A carrier mixture of plurolic/tween 80 was prepared by mixing 15 g of plurolic F68 and 30 g of tween 80 on a stirrer at 400 rpm for 2 hours at 60° C.

A homogeneous mixture was formed by mixing 15 g of the dispersal phase and 37.5 g of the carrier mixture on a stirrer at 1000 rpm, for 5 hours at 60° C.

Removal of insoluble substances in the mixture was carried out by high-speed centrifugation of 10000 rpm, for 20 minutes at room temperature.

The resulting mixture was left overnight, subsequently homogenized in a homogenizing stirrer at 3000 rpm for 1 hour and repeated 5 times; and

The homogeneous mixture obtained after the homogenization was filtered by a nanofiltration system to obtain the nano-microemulsion system of ferulic.

### Example 2: Characteristics of the Pre-Nanoemulsion System

The resulting product was tested with modern analytical methods. The results of analysis are as follows:

### Results of HR-TEM

The sample of aqueous solutions of ferulic was analyzed by high resolution transmission electron microscopy (HR-TEM) JEM 2100. The results of HR-TEM showed the self-microemulsifying systems of ferulic were highly dispersed in water, containing spherical nanoparticles of ferulic with diameter of about 100-200 nm.

### Results of Analysis of Physicochemical and Microbiological Standards

The nano samples of ferulic after the production were tested in relation to microbiological and physicochemical standards. The results are shown in the table below:

| # | Standards | Unit | Test method | Result |
|---|---|---|---|---|
| 10.1 | Total aerobic | CFU/mL | ISO 4833-1:2013 | KPH |
| | microorganisms | | | (LOD: |
| | | | | 1 CFU/mL) |
| 10.2 | Cl. Perfringens | CFU/mL | TCVN 4991:2005 | KPH |
| | | | | (LOD: |
| | | | | 1 CFU/mL) |
| 10.3 | Coliforms | CFU/mL | TCVN 6848:2007 | KPH |
| | | | | (LOD: |
| | | | | 1 CFU/mL) |
| 10.4 | E. coli | CFU/mL | TCVN | KPH |
| | | | 7924 -2:2008 | (LOD: |
| | | | | 1 CFU/mL) |
| 10.5 | Total yeast- | | TCVN 8275-1:2010 | KPH |
| | mold spores | | | (LOD: 1 |
| | | | | CFU/mL) |
| 10.6 | Cadmium | mg/kg | H.HD.QT.429 | KPH |
| | content | | (ICP-MS) | (LOD: |
| | | | | 0.004 mg/kg) |
| 10.7 | Lead content | mg/kg | H.HD.QT.429 | 0.037 |
| | | | (ICP-MS) | |
| 10.8 | pH | — | H.HD.QT.070 | 7.29 |
| 10.9 | Density | g/mL | NIFC.05. M. 197 | 1.076 |

### Advantageous Effects of the Invention

The proposed process for production a nano-microemulsion system of ferulic succeeds in manufacturing the nano-microemulsion system having ferulic nanoparticles with uniformity and good solubility in water.

The substances used in the process for production a nano-microemulsion system of ferulic are highly dispersed in water with high safety, non-toxicity and few side-effects, the nano-microemulsion system of ferulic obtained by the present invention has therefore high safety when being used.

The process of the present invention is simple, easy to perform and suitable with the currently domestic conditions of practice.

What is claimed is:

1. A process for production of a nano-microemulsion system of ferulic acid includes the steps of:
a) preparing a dispersal phase by dissolving ferulic acid in PEG-40 Hydrogenated Castor Oil (acrysol K140) in a ratio (g/g) of ferulic/PEG-40 Hydrogenated Castor Oil of 1:4 on a stirrer at 500 rpm for 45 minutes at 40° C. to obtain the ferulic dispersal phase;
b) preparing a carrier mixture of plurolic/tween 80 by mixing Polyoxyethylene-polyoxypropylene block copolymer (plurolic F68) and tween 80 in a ratio (g/g) of plurolic F68/tween 80 of 1:2 on a stirrer at 400 rpm for 2 hours at 60° C.;
c) forming a homogeneous mixture by mixing the ferulic dispersal phase and the carrier mixture of plurolic/tween 80 so that the substances form the homogeneous mixture having a ratio (g/g) of the dispersion/the carrier mixture is 2:5 on a stirrer at 1000 rpm, for 5 hours at 60° C.;
d) removing insoluble substances in the mixture by high-speed centrifugation of 10000 rpm, for 20 minutes at room temperature;
e) leaving the mixture obtained in step d) overnight, subsequently homogenizing in a homogenizing stirrer at 3000 rpm for 1 hour and repeating 5 times; and
f) filtering the homogeneous mixture obtained after the homogenization by a nanofiltration system to obtain the nano-microemulsion system of ferulic.
